(19) United States
(12) Patent Application Publication
(10) Pub. No.: US 2006/0248615 A1
(43) Pub. Date: Nov. 2, 2006

(54) Synthetic spider silk proteins and expression thereof in transgenic plants

(76) Inventors: Jurgen Scheller, Kiel (DE); Udo Conrad, Hausneindorf (DE); Frank Grosse, Jena (DE); Karl-Heinz Guehrs, Jena (DE)

Correspondence Address: KENYON & KENYON LLP, 1500 K STREET N.W., SUITE 700, WASHINGTON, DC, 20005, US

(21) Appl. No.: 10/297,389

(22) PCT Filed: Jun. 11, 2001
(86) PCT No.: PCT/EP01/06586

§ 371 Date: May 30, 2003

(30) Foreign Application Priority Data
Jun. 9, 2000 (DE) 100 28 212.1
Oct. 24, 2000 (DE) 100 53 478.3
Mar. 21, 2001 (DE) 101 13 781.8

(51) Int. Cl. C12N 15/82; C12N 5/04; A01H 1/00

(52) U.S. Cl. 800/288; 435/468; 435/419; 530/353

(57) ABSTRACT

The invention relates to a DNA sequence coding for a synthetic protein and recombinant spider silk proteins which are coded by the inventive DNA sequence. The invention also relates to methods for producing plants or plant cells containing the recombinant spider silk protein and transgenic plants and cells containing a DNA sequence coding for a synthetic spider protein. The invention further relates to a method for obtaining a vegetable spider silk protein from transgenic plants in addition to vegetable spider silk proteins produced according to said method.

### RELATED APPLICATIONS

This is the national phase of PCT/EPO/06586 filed Jun. 11, 2001, which claim priority to DE 100 28 212.1 filed Jun. 9, 2000, DE 100 53 478.3 filed Oct. 24, 2000 and DE 101 13 781.8 filed Mar. 21, 2001, the entire contents of which are incorporated.

The invention relates to a DNA sequence that codes for a synthetic spider silk protein, recombinant spider silk proteins coded by the DNA sequence according to the invention, methods of producing plants or plant cells containing recombinant spider silk protein, as well as transgenic plant cells and plants containing a DNA sequence that codes for a synthetic spider silk protein. In addition, the invention relates to a method of obtaining plant spider silk protein from transgenic plants, as well as plant spider silk proteins produced according to said method.

Spider silk exhibits outstanding mechanical properties that are superior to those of many known natural and synthetic materials. The main constituents of spider silk are fibre proteins, e.g., fibroin, from the silkworm, as well as spidroin 1 and spidroin 2 from Nephila clavipes. The strength and elasticity of the silk are based on the presence of short, repetitive amino acid units within these natural proteins. These mechanical properties predestine the spider silk for a series of the most varied technical applications, e.g., the manufacture of stable threads or silks. In addition, due to their protein chemical properties the spider silk threads have a low immunogenic and allergenic potential, so that, when combined with their mechanical

properties, these threads can be beneficially used in medicine, e.g., as a natural yarn for closing wounds, as adhesion surfaces for cultivated cells, as frames for artificial organs and the like.

However, one prerequisite for such technical or medical use of the spider silk is the large-scale production of spider threads or spider silk proteins. To this end, attempts have been made up to now to express the spidroin or fibroin genes responsible for the production of the spider silk in E. coli. However, during reproduction in bacteria the frequently repeated sequences in the corresponding genes are gradually lost. Another problem is the quantity of genetic information, which appears to be too extensive for the bacterium, so that a complete readout of the spider silk genes is not always possible.

While expression experiments in yeast cells yielded more stable and longer silk proteins, the threads spun from them do not exhibit the same advantageous properties of natural silk, so that such synthetically produced silk cannot be used for example for medical purposes. There is thus a need for synthetic silk proteins that can be produced on an industrial scale which after spinning into threads display mechanical properties comparable with those of natural silk.

Therefore, the object of the present invention is to provide DNA sequences that code for a synthetic spider silk protein as similar as possible to the previously known natural sequences of fibre proteins in spider silk. In addition, the object of this invention is to provide a method according to which synthetic spider silk proteins can be produced on a large-scale.

The object of the invention is also to provide DNA sequences that code for a synthetic spider silk protein exhibiting the advantageous and desirable properties of native spider silk protein, but where the range of properties of the native protein has additionally been modified or optimised in this way or that, depending on the intended application.

Other objects of this invention will become clear from the following description.

The above objects are achieved by the features in the independent claims.

Advantageous embodiments are described in the sub-claims.

The DNA sequence disclosed by the present invention codes for a synthetic fibre protein, in particular a synthetic spider silk protein exhibiting a homology of at least 80%, preferably of at least 84%, more preferably of at least 88%, especially preferably of at least 90% and 92%, and most preferably of at least 94% with spidroin and/or fibroin proteins, in particular with the spidroin 1 protein, especially preferably with the spidroin 1 protein from Nephila clavipes.

Within the context of this invention, homology denotes similarity between amino acid sequences based on identical or homologous amino acid structural units. The person skilled in the art knows which amino acids are to be regarded as homologous, e.g., (i) isoleucine, leucine and valine among each other, (ii) asparagine and glutamine, (iii) aspartic acid and glutamic acid.

The DNA sequence according to the invention is composed of modules comprising a group of successively arranged oligonucleotide sequences, wherein the oligonucleotide sequences each

code for repetitive units from spidroin and/or fibroin proteins.

The structure of the inventive DNA sequence composed of various modules, which are in turn made out of different short amino acid repeats typical for spidroins or fibroins, whereby the principle of successively arranging the corresponding oligonucleotide sequences or modules is oriented towards natural spidroin and/or fibroin sequences, ensures a very high homology to previously known natural spidroin or fibroin sequences. This ensures that the spider silk proteins coded by the DNA sequence according to the invention after being spun into threads will exhibit outstanding mechanical properties in terms of their strength and elasticity, which are comparable to the mechanical properties of natural spider threads.

In addition, the modular structure of the DNA sequence according to the invention makes it possible to modify the synthetic genes quite simply by means of genetic engineering, so that multimers of synthetic spider silk proteins of any size can be produced as desired. Further, the spider silk proteins coded by the DNA sequence according to the invention can, due to their modular structure, be fused with other fibre protein sequences. One special advantage of the DNA sequence of the present invention is that due to its modular structure it is easy to fuse with sequences that code for purifying elements or solubility-altering peptides.

The invention also relates to DNA sequences that code for a synthetic spider silk protein and which are comprised of modules comprising a group of successively arranged oligonucleotide sequences, whereby each of the oligonucleotide sequences codes for repetitive units from spidroin proteins and the modules are freely arranged, the free arrangement making it possible for synthetic spider silk protein to exhibit an altered range of properties compared to native spider silk protein.

Therefore, the invention makes it possible, for the first time, to synthesize new types of silk proteins based on modular structured silk protein genes, the new types of silk proteins having a modified range of properties compared to native silk protein, while at the same time containing the essential structural determinants of naturally occurring silk proteins. While maintaining the essential structural sections of natural silk proteins, which are combined with each other in a novel manner according to the invention, synthetic silk proteins are provided which, with regard to their elasticity, tensile strength, solubility behaviour, heat and acid resistance and swelling capacity, are modified or optimised in a particular way depending on the particular purpose.

Specific arrangements of the obtained synthetic proteins can make the obtained protein particularly well suited for a specific purpose. As an alternative, of course, one can screen for a protein particularly suited for a specific application, e.g. having increased elasticity compared to native protein. Increased elasticity may be achieved by purposely using more elastic modules for the structure instead of rigid modules.

In any event, the combination of properties, which makes the recombinant spider silk proteins according to the invention so useful and attractive from a material/technical point of view, can be influenced within desired limits by the arrangement of the modules, without differing too much from the attractive range of properties of the natural protein.

The gene cassette with the highest homology to the cDNA isolated from the native host, called SO1, exhibits the following combination of structural sections designated as a module (represented by various letters):

H_B_C_B_C_G_D_C_G_D_C_B_C_B_B_G_D_B_C

(see also FIG. 3). In contrast to the approaches in the prior art with respect to spider silks and natural silks, the teaching of the present invention for assembling the gene cassettes allows a new and targeted arrangement of these modules in a completely variable manner. This makes it possible to create completely new types of proteins, and also to reconstruct the naturally occurring protein. In addition to the module sequence series shown above for the naturally occurring sequence, any number of variations in any scheme are thus now possible, such as the following, each of which yield proteins having different properties:
Hₙ≠Bₙ≠Cₙ≠Dₙ≠(HₓB_{y})ₙ≠(HₓC_{y})ₙ≠ . . . ≠(HᵢBⱼCₖDₗ)ₙ.
Embodiments for the possibilities of creating such structures and for the different properties of the resulting proteins can be gathered from the examples provided below.

In addition to the properties already mentioned, which can be further modified or optimised, additional RGD sequences, for example, may be used to achieve an enhanced adhesion of cells (Massia et al. (2001), J. Biomed. Mater. Res. 56: 390-399). Other useful properties of the synthetic spider silk proteins according to the invention also may be derived from the following description and examples.

In a particularly preferred embodiment of this invention, the spider silk protein coded by the DNA sequence according to the invention has a homology of at least 84%, preferably of at least 90%, and especially preferably of at least 94% with the spidroin 1 protein from Nephila clavipes. Spidroin 1 from Nephila clavipes is significantly involved in the structure of a support thread that is mechanically particularly stable and elastic.

The modular structure of the DNA sequence according to the invention renders it possible to construct genes that encode very large spider silk proteins, wherein the high degree in homology with spidroin and/or fibroin proteins, in particular with spidroin 1, especially preferably with spidroin 1 from Nephila clavipes, is always retained. The size distribution achievable in this way for the proteins coded by the DNA sequences according to the invention corresponds to the range of spider silk proteins that can be observed after dissolving natural spider silk. This identical range of sizes as well the high sequence homology defines the synthetic genes according to the invention as genes that code for spider silk proteins. In contrast to natural spider silk, which consists of a mixture of spider silk proteins, this invention provides spider silk protein genes that represent a gene class by having high homology, and permit simple gene-technological manipulation.

The modules for assembling the DNA sequence of the present invention comprise a group of successively arranged oligonucleotide sequences, which preferably are selected from the group consisting of:

(SEQ ID NO: 1)

a) TATGAGCGCTCCCGGGCAGGGT;

(SEQ ID NO: 2)

b) AGCTTTTAGGTACCAATATTAATCTGGCCGGCTCCACC;

(SEQ ID NO: 3)

c) TATGGTCTGGGG;

(SEQ ID NO: 4)

d) GGCCAGGGTGCTGGCCAA;

(SEQ ID NO: 5)

e) GGTGCAGGAGCWGCWGCWGCWGCTGCAGGTGGA;

(SEQ ID NO: 6)

f) GCCGGCCAGATTAATATTGGTACCTAAA;

(SEQ ID NO: 7)

g) CTGCCCGGGAGCGCTCA;

(SEQ ID NO: 8)

h) ACCACCATAACCTCC;

(SEQ ID NO: 9)

i) AGCACCCTGGCCCCCCAG;

(SEQ ID NO: 10)

j) TGCAGCWGCWGCWGCWGCTCCTGCACCTTGGCC;

(SEQ ID NO: 11)

k) TATGAGATCTGGCCAAGGAGGT;

(SEQ ID NO: 12)

l) TTGGCCAGATCTCA;

(SEQ ID NO: 13)

m) AGTCAGGGTGCTGGTCGTGGAGGCCAA;

(SEQ ID NO: 14)

n) TCCACGACCAGCACCCTGACTCCCCAG;

(SEQ ID NO: 15)

o) AGTCAGGGCGCTGGTCGTGGGGGACTGGGTGGCCAA;

(SEQ ID NO: 16)

p) ACCCAGTCCCCCACGACCAGCGCCCTGACTCCCCAG;

(SEQ ID NO: 17)

q) CTGGGAGGGCAGGGAGCGGGCCAA;

(SEQ ID NO: 18)

r) CGCTCCCTGCCCTCCCAGACCTCC;
and

s) sequences that exhibit at least 80%, preferably
at least 90%, especially preferably at least 94%
sequence identity to the sequences of a) to r).

The modules preferably comprise at least four oligonucleotide sequences, which preferably differ, in order to mimic the natural spider silk proteins in an authentic manner. The DNA sequence according to the invention in turn is preferably composed of at least four of the modules described above.

The structure of the DNA sequence according to the invention is described below by way of example. First of all, the oligonucleotides shown in FIG. 1 are prepared, which code for amino acid sequences corresponding to spidroin-typical, short amino acid repeats. These oligonucleotides are combined with each other using gene technological methods, the combination being geared towards the natural spidroin sequence (see FIG. 2). Modules A, B, C, D, E and F obtained in this way are again combined with each other (see FIG. 3). In this way, DNA sequences according to the invention are provided, which exhibit a homology of at least 85%, preferably of at least 90%, and particularly preferably of at least 94% with spidroin proteins at the amino acid level.

In a further embodiment, the DNA sequence according to the invention comprises in addition to the modules described above nucleic acid sequences that code for repeated units from fibroin proteins, preferably from the fibroin protein of the silkworm.

Sequences SEQ ID NO: 19 to 29 exhibit especially preferred DNA sequences according to the invention.

In addition, the invention has surprisingly succeeded for the first time in creating synthetic spider silk proteins in transgenic plants. In this way, synthetic spider silk proteins can be produced on a large scale. To ensure stable expression of the DNA sequence according to the invention in plants, a recombinant nucleic acid molecule is provided that comprises the DNA sequence according to the invention described above, as well as an ubiquitously acting promoter, preferably the CaMV 35S promoter. The provision of the recombinant nucleic acid molecule according to the invention permits the expression and accumulation of synthetic spidroin or fibroin sequences in transgenic plants.

To ensure that the DNA sequence according to the invention is expressed and accumulated in suitable compartments of transgenic plants, the nucleic acid molecule according to the invention comprises, in addition to the DNA sequence according to the invention and the ubiquitously acting promoter, preferably at least one nucleic acid sequence that codes for a plant signal peptide.

In a preferred embodiment, the endoplasmatic reticulum (ER) is the selected compartment for the expression or accumulation of the synthetic spider silk protein. This compartment is particularly suitable for stable the accumulation of foreign proteins in plants. To ensure transport into the ER, the nucleic acid molecule according to the invention preferably comprises corresponding signal peptides, the LeB4Sp sequence being particularly preferred.

ER retention, if desired, is ensured according to the invention in that the nucleic acid molecule according to the invention additionally comprises a nucleic acid sequence coding for an ER retention peptide. Retention in the ER is preferably achieved by the amino acid sequence KDEL (SEQ ID NO: 52) attached to the C terminus.

In addition, it may be advantageous to place the DNA sequence according to the invention at the plasmalemma, i.e., the cell membrane. For this reason, in an alternative embodiment the recombinant nucleic acid molecule according to the invention comprises the DNA sequence according to the invention fused with the N terminus of a transmembrane domain. Preferably, this transmembrane domain is the transmembrane domain of the PDGF receptor, the so-called HOOK sequence (see FIG. 4).

In a especially preferred embodiment of this invention, the nucleic acid molecule according to the invention is fused with ELPs (elastin-like polypeptides). ELPs are oligomeric repeats of the pentapeptide Val-Pro-Gly-Xaa-Gly ((SEQ ID NO: 53), wherein Xaa is every amino acid except proline and is preferably Gly), and are subjected to a reversible inverse temperature transition. They are very soluble in water below the inverse transition temperature (T₁), but have a sharp phase transition state in the range of 2° C. to 3° C., when the temperature is increased to above T₁, which leads to precipitation and aggregation of the polypeptide. D. E. Meyer and A. Chilkoti, Nat. Biotech. 1999, 17: 1112-1115, have described that ELP fusions with recombinant proteins alter the solubility behaviour of these recombinant proteins at various temperatures and concentrations in a targeted fashion. In the present invention, this is used to establish purification strategies described in detail below for the spider silk protein coded by the DNA sequence according to the invention. Preferably, the ELPs coded by the nucleic acid sequence in the nucleic acid molecule according to the invention comprise from 10 to 100 of the pentameric units described above (see FIG. 5).

The chimeric gene constructs or recombinant nucleic acid molecules described above are produced using conventional cloning techniques (see for example Sambrook et al. (1989), Molecular Cloning: A Laboratory Manual, 2^{nd} edition, Cold Spring Harbour Laboratory Press, Cold Spring Harbour, New York). These typical molecular biological techniques make it possible to prepare or produce desired constructs for the transformation of plants. Methods for cloning, mutagenesis, sequence analysis, restriction analysis and other additional biochemical/molecular biological methods commonly used for gene technologically manipulating prokaryotic cells are well known to the person skilled in the art. Thus, it is not only possible to produce suitable chimeric gene constructs containing the respectively desired fusion of promoters, DNA sequence according to the invention, sequence coding for a plant signal peptide, sequence coding for an ER retention peptide, sequence coding for a transmembrane domain and/or sequences coding for purifying elements or solubility-altering peptides, but rather the person skilled in the art may use routine techniques to introduce various mutations or deletions into the respective genes, if desired.

The invention also relates to vectors and microorganisms that contain nucleic acid molecules according to the invention, and whose use renders possible the production of plant cells or plants that produce spider silk proteins. These vectors include in particular plasmids, cosmids, viruses, bacteriophages and other vectors common in genetic engineering. The microorganisms are primarily bacteria, viruses, fungi, yeasts and algae.

Since the DNA sequences according to the invention, because of their repetitive nature, exhibit hardly any unique restriction sites, the vectors according to the invention or the genes encoding the synthetic spider silk protein were adapted accordingly using various strategies (see FIGS. 6 to 8). When the DNA sequences according to the invention are amplified by PCR, preferably oligonucleotides are first ligated thereto due to the extremely repetitive nature of the DNA sequences according to the invention, which then serve as templates for the subsequent PCR reactions (see FIG. 7).

Furthermore, the present invention provides a recombinant spider silk protein that is coded by the DNA sequence according to the invention. This synthetic spider silk protein according to the invention, preferably having a molecular weight ranging from 10 to 160 kDa, exhibits a homology of at least 85%, preferably of at least 90%, and particularly preferably of at least 94% with spidroin and/or fibroin proteins. This high degree of homology with the natural fibre proteins of the spider and silkworm ensures that the outstanding mechanical properties of the natural spider threads are achieved when the proteins according to the invention are spun into threads.

In addition, the proteins according to the invention surprisingly exhibit novel physicochemical properties. For example, the solubility of these synthetic fibre proteins according to the invention is sustained extremely well in aqueous solutions, even after prolonged boiling. In conjunction with the also occurring solubility in organic solutions and the precipitation behaviour in the presence of high salt concentrations, these new properties of the synthetic spider silk proteins according to the invention may therefore be used to develop technically feasible extraction and purification techniques. These properties are enhanced even further if the synthetic spider silk proteins according to the invention are specifically accumulated in specific compartments, in particular in the ER of transgenic plants.

Examples of amino acid sequences of the recombinant synthetic spider silk proteins according to the invention are the sequences identified in SEQ ID NO: 30 to 40. Alternatively, the spider silk proteins according to the invention may also be synthesized according to chemical methods known to the person skilled in the art, although recombinant manufacture is preferred.

The invention also relates to a method for manufacturing spider silk protein-producing plants or plant cells, comprising the following steps:
a) Manufacture of a recombinant nucleic acid molecule according to the invention as described above,
b) Transfer of the nucleic acid molecule from a) to plant cells; and
c) optionally, regeneration of fertile plants from the transformed plant cells.

In addition, the invention relates to plant cells containing the nucleic acid molecules according to the invention or the vector according to the invention. The invention also concerns harvest products and propagating material of transgenic plants, as well as the transgenic plants thereof, which contain a nucleic acid molecule according to the invention.

To prepare the introduction of foreign genes into higher plants, or their cells, a large number of cloning vectors are available which contain a replicating signal for E. coli and a marker gene for selecting transformed bacterial cells. Examples of such vectors are pBR322, pUC series, M13 mp series, pACYC184 etc. The desired sequence may be introduced into the vector at a suitable restriction site. The resulting plasmid is then used for the transformation of E. coli cells. Transformed E. coli cells are cultivated in a suitable medium and then harvested and lysed, and the plasmid is recovered. The analytic methods used to characterise the produced plasmid DNA generally include restriction analyses, gel electrophoreses and other biochemical and molecular biological methods. After each manipulation step the plasmid DNA may be cleaved and the obtained DNA fragments may be linked to other DNA sequences.

A plurality of techniques is available for introducing DNA into a plant host cell, and the person skilled in the art will not have any difficulties in selecting a suitable method in each case. These techniques comprise the transformation of plant cells with T-DNA by use of Agrobacterium tumefaciens or Agrobacterium rhizogenes as the transforming agent, the fusion of protoplasts, injection, electroporation, the direct gene transfer of isolated DNA into protoplasts, the introduction of DNA by means of biolistic methods as well other possibilities that have been well established for several years and belong to the normal repertoire of the person skilled in the art of plant molecular biology or plant bioengineering.

For injection and electroporation of DNA in plant cells, no special requirements are imposed per se on the used plasmids. The same applies to direct gene transfer. Simple plasmids, such as pUC derivatives can be used. However, if entire plants are to be regenerated from these transformed cells, the presence of a selectable marker gene is recommended. The person skilled in the art is familiar with current selection markers, and he would have no problem choosing a suitable marker.

Depending on the method for introducing desired genes into the plant cell, additional DNA sequences may be required. If, for example, the Ti or Ri plasmid is used for the transformation of the plant cell, at least the right border, however more often both the right and left border of the T-DNA contained in the Ti or Ri plasmid, respectively, must be linked to the genes to be integrated as a flanking region. If agrobacteria are used for the transformation, the DNA to be integrated must be cloned into special plasmids, and specifically either into an intermediate or into a binary vector. The intermediate vectors can be integrated into the Ti or Ri plasmid of the agrobacteria via homologous recombination due to sequences that are homologous to sequences in the T-DNA. This plasmid also contains the vir-region, which is required for the T-DNA transfer. Intermediate vectors cannot replicate in agrobacteria. A helper plasmid can be used to transfer the intermediate vector to Agrobacterium tumefaciens (conjugation). Binary vectors can replicate both in E. coli and in agrobacteria. They contain a selection marker gene and a linker or polylinker, which are framed by the right and left T-DNA border region. They can be transformed directly into the grobacteria. The agrobacterial host cell should contain a plasmid carrying a vir-region. The vir-region is necessary for transferring the T-DNA into the plant cell. Additional T-DNA can be present. The agrobacterium transformed in this way is used to transform plant cells. The use of T-DNA for the transformation of plant cells has been intensively studied and sufficiently described in generally known articles and manuals for plant transformation. Plant explants can be specifically cultivated with Agrobacterium tumefaciens or Agrobacterium rhizogenes for the transfer of DNA into the plant cells. Whole plants can then be regenerated from the infected plant material (e.g., leaf parts, stem segments, roots, but also protoplasts or suspension-cultivated plant cells) in a suitable medium that can contain antibiotics or biocides for the selection of transformed cells.

Once the introduced DNA has been integrated into the genome of the plant cell, it is generally stable there, and is maintained in the progeny of the originally transformed cell as well. It normally contains a selection marker, which makes the transformed plant cells resistant to a biocide or an antibiotic such as kanamycin, G 418, bleomycin, hygromycin, methotrexate, glyphosate, streptomycin, sulfonylurea, gentamycin or phosphinotricine, etc. Therefore, the individually selected marker should allow the selection of transformed cells from cells lacking the introduced DNA. Also suited for this purpose are alternative markers, such as nutritive markers, screening markers (e.g., GFP, green fluorescent protein). Naturally, selection markers need not be used at all, although this would involve a fairly high screening expenditure. If marker-free transgenic plants are desired, the person skilled in the art also has strategies at his disposal that enable subsequent removal of the marker gene, e.g., cotransformation, sequence-specific recombinases.

The transgenic plants are regenerated from transgenic plant cells by usual regeneration methods using known nutrient media. The plants obtained in this way can then be analysed for the presence of the introduced nucleic acid encoding a synthetic spider silk protein using conventional methods, including molecular biological methods such as PCR and blot analyses.

The transgenic plant or transgenic plant cell can be any desired monocotyledonous or dicotyledonous plant or plant cell.

Useful plants or cells from useful plants are preferred. Especially preferred are transgenic plants selected from the group consisting of the tobacco plant (Nicotiana tabacum) and the potato plant (Solanum tuberosum).

The expression of the synthetic spider silk protein according to the invention in the plants according to the invention or plant cells according to the invention can be detected and followed using conventional molecular biological and biochemical methods. The person skilled in the art knows these techniques and he can easily select a suitable detection method without any problem, e.g., a Northern blot analysis or a Southern blot analysis.

FIG. 9 shows an example for the manufacture of transgenic spider silk protein-producing plants. The PCR-amplified sequences can possibly contain frame shift mutations. For this reason, the sequences according to the invention must be tested prior to the generation of transgenic plants. Performing a sequence analysis each starting from the flanking vector sequences can do this. Longer constructs of more than 1 kb cannot be verified in this way, since due to the repetitive properties of the DNA sequences according to the invention internal sequencing primers provide no reliable sequences that can be evaluated accurately. For this reason, amplified spidroin sequences were preferably cloned into the bacterial expression vector pet23a (Novagen, Madison, USA). By immunodetection of the expression frame shift mutations may then be precluded.

The nucleic acid molecules or expression cassettes according to the invention are usually cloned as HindIII fragments into shuttle vectors such as pBIN, pCB301 and/or pGSGLUC1. These shuttle vectors are preferably transformed in Agrobacterium tumefaciens. The transformation of Agrobacterium tumefaciens is usually verified via Southern blot analysis and/or PCR screening.

The invention also relates to propagating material and harvest products of the inventive plants, e.g., fruits, seeds, bulbs, tubers, seedlings, cuttings, etc.

Further, the invention relates to a method of obtaining plant spider silk protein, comprising the following steps:
a) transfer of a recombinant nucleic acid molecule or vector according to the invention containing a DNA sequence that codes for a synthetic spider silk protein to plant cells;
b) optionally, regeneration of plants from the transformed plant cells;
c) processing of the plant cells from a) or plants from b) to obtain plant spider silk protein.

In another important aspect of this invention, methods of obtaining recombinant manufactured spider silk proteins are provided that comprise the transfer of an inventive recombinant nucleic acid molecule or vector containing a DNA sequence that codes for a synthetic spider silk protein to any cells, i.e. for example bacterial or animal cells in addition to plant cells. An essential characteristic of these methods according to the invention is the purification step of the recombinantly manufactured spider silk proteins, which among other things utilize the proteins' special properties vis-à-vis solubility when heated and/or when acid is added.

In one embodiment of the method according to the invention, the recombinantly manufactured spider silk protein is purified by heat-treating the cell extract, e.g., a plant seed extract, and subsequently separating the denatured proteins naturally occurring in the cell, e.g. the native proteins of the plant, for example by centrifugation. In this case, the beneficial feature of the recombinantly produced spider silk proteins is utilized, namely that the proteins maintain solubility when aqueous solutions are heated up to boiling point. In contrast, synthetic fibre proteins of the spider and silkworm after expression in Pichia pastoris only remain in a dissolved status when heated up to a temperature of 63° C., and then only for 10 minutes.

In another embodiment of the method according to the invention of obtaining recombinantly manufactured spider silk proteins, purification is performed by adjusting an acidic pH by adding acid, preferably hydrochloric acid, to the cell extract, for example to the plant extract.

The acidic pH, particularly a pH ranging from 1.0 to 4.0, more preferably ranging from 2.5 to 3.5, most preferably a pH of 3.0, is here maintained preferably for several minutes, more preferably for about 30 minutes, at a temperature below room temperature, preferably approximately 4° C. Again, an unexpected property of the proteins obtained by the method of the invention is exploited, namely that they remain in solution during acidification specifically up to a pH of 3.0 at 4° C. On the other hand the proteins naturally occurring in the cell, for example proteins that are produced naturally in the cell, are precipitated by this treatment and are then separated, especially by centrifugation.

The above-described solubility properties of the spider silk proteins that are recombinantly produced according to the invention are very surprising, were not foreseeable in this form, and permit an efficient, fast and inexpensive purification procedure when extracted from cells, in particular plant cells.

In another embodiment of the method according to the invention, a nucleic acid molecule that additionally comprises a nucleic acid sequence coding for ELPs is transferred to the cells. In this case the purification of the recombinantly manufactured spider silk protein is performed as follows: in a first step, the spider silk-ELP fusion protein is enriched by heat-treating the crude extract. Surprisingly, the fusion proteins retain the excellent solubility of the spider silk proteins at high temperatures. The bulk of the proteins naturally occurring in the cells are precipitated during this temperature increase. In the next step, further increasing the temperature, preferably to a temperature of at least 60° C., precipitates the spider silk-ELP fusion proteins. Precipitation preferably takes place in the presence of a suitable salt concentration, e.g. a NaCl concentration of at least 0.5 M, preferably in a range of from 1 M to 2 M. Finally, the ELP fragment is cleaved, preferably via digestion with CNBr.

Through the method for obtaining recombinantly manufactured spider silk protein according to the invention described above, the proteins in plants may be accumulated to high concentrations, preferably up to an expression level of about 4% of the total soluble protein. Thus, for the first time, methods are provided that can be used for technically feasible enrichment of recombinant spider silk protein.

In another aspect of the present invention, the spider silk proteins according to the invention can be used to produce synthetic threads, as well as films and membranes. Such products are especially suitable for medical applications, in particular for closing wounds and/or as frames or covers for artificial organs. Further, the films and membranes made out of the spider silk proteins according to the invention can be used as adhesion surfaces for cultivated cells, as well as for filtering purposes.

This invention will be explained in the following examples, which serve merely to illustrate the invention, and are in no way to be understood as restrictive.

### EXAMPLES

### Example 1

### Expression and Stable Accumulation of Synthetic Fibre Proteins of the Spider and Silkworm in the Endoplasmatic Reticulum of Leaves or Tubers from Transgenic Tobacco and Potato Plants

FIGS. 10a and b show the amino sequences of synthetic spider silk proteins having a high degree of homology with the spidroin 1 protein from Nephila clavipes, the C-terminal and non-repetitive constant region not being shown. These synthetic spider silk proteins consist of modules, which in turn comprise successively arranged oligonucleotide sequences. The combination of several modules resulted in the assembly of the various synthetic genes, wherein mixed forms with sequences based on fibroin 1 have also been created.

Table 1 below lists various plant expression cassettes, which code for various synthetic fibre proteins according to the invention with the sequences SEQ ID NO: 30 to 40.

TABLE 1

| | | Calculated | |
|---|---|---|---|
| Plant | Number of amino | molecular weight | |
| expression | acids (with | (with leader | |
| cassette | leader sequence) | sequence) | Homology |
| SB1 | No. 1 - 149 AS | 11 kDa | spidroin 1 |
| (SEQ ID No. 19) | | | |
| SD1 | No. 2 - 182 AS | 13 kDa | spidroin 1 |
| (SEQ ID No. 21) | | | |
| SA1 | No. 3 - 215 AS | 16 kDa | spidroin 1 |
| (SEQ ID No. 26) | | | |
| SE1 | No. 4 - 275 AS | 20 kDa | spidroin 1 |
| (SEQ ID No. 20) | | | |
| SF1 | No. 5 - 317 AS | 24 kDa | spidroin 1 |
| (SEQ ID No. 29) | | | |
| SM12 | No. 6 - 410 AS | 31 kDa | spidroin 1 |
| (SEQ ID No. 28) | | | |
| SO1 | No. 7 - 676 AS | 52 kDa | spidroin 1 |
| (SEQ ID No. 27) | | | |
| SO1SM12 | No. 8 - 1035 AS | 82 kDa | spidroin 1 |
| (SEQ ID No. 23) | | | |
| SO1SO1 | No. 9 - 1301 AS | 102 kDa | spidroin 1 |
| (SEQ ID No. 22) | | | |
| SO1SO1SO1 | No. 10 - 1926 AS | 151 kDa | spidroin 1 |
| (SEQ ID No. 24) | | | |
| FA2 | No. 11 - 264 AS | 20 kDa | spidroin 1 |
| (SEQ ID No. 25) | | | and fibroin |

The target-specific transport and accumulation of the sequences according to the invention in the endoplasmatic reticulum of cells of transgenic plants was achieved by an N-terminal signal peptide sequence and a C-terminal ER retention sequence (KDEL, SEQ ID NO: 52). A detection sequence in the form of a c-myc-tag at the C-terminal end of the transgenic synthetic fibre proteins permits the detection of transgenic products in plant extracts.

Cassettes SO1 and FA2 are shown in detail as examples in FIGS. 10a and 10b. The plant expression cassettes SB1, SD1, SA1, SE1, SF1, SM12, SO1SM12, SO1SO1 and SO1SO1SO1 were created according to the same structural principle. Varying the basic module repeats results in synthetic fibre proteins containing a different number of amino acids and correspondingly different molecular weight (see Table 1).

FIG. 2 describes schematically how the constructs mentioned above are arranged. The SmaI and NaeI restriction sites were introduced for directly cloning the synthetic fibre protein genes of the present invention. To this end, a PCR product containing the corresponding restriction sites was cloned with the primer combination 5′-pRTRA-SmaI and 3′-pRTRA-NotI in the plasmid pRTRA ScFv SmaI/BamHIΔ via BamHI and NotI. Synthetic fibre protein genes were cloned from the fibre protein gene derivatives of plasmids 9905 or 9609 in vector pRTRA.7/3 placeholder. Selection of restriction endonuclease recognition sequences at the 5′- and 3′-end of the synthetic fibre protein genes (SmaI and NaeI) allows them to be freely combined with each other, and larger fibre protein genes can be assembled in one cloning step according to the invention.

In this way, transgenic synthetic spider silk proteins were accumulated to high concentrations in the endoplasmatic reticulum of transgenic tobacco and potato plants (see FIGS. 12a and 12b). Table 2 shows the maximal accumulation level of synthetic spider silk proteins according to the invention in the ER of leaves of transgenic tobacco and potato plants. The enrichment of transgenic synthetic fibre proteins was estimated by means of a comparison with transgenic recombinant antibodies, which were likewise provided with the same tag. Thus for the first time, an accumulation of spider silk proteins in plants is described using potato and tobacco as an example.

TABLE 2

| | Fibre protein | | | |
|---|---|---|---|---|
| | SD1 | SM12 | SO1 | FA2 |
| Tobacco | | | | |
| Accumulated amount in | ˜0.5% | ˜0.5% | ˜0.5% | ˜0.5% |
| percentage of total protein | | | | |
| Potato | | | | |
| Accumulated amount in | ˜0.5% | ˜0.5% | ˜0.5% | ˜0.5% |
| percentage of total protein | | | | |

A defined quantity of the fibre protein-containing total protein extract (40 μg) and a defined quantity of a reference protein with c-myc-immunotag (50 ng ScFv) were separated via SDS gel electrophoresis, and synthetic fibre proteins and reference proteins were detected in a Western blot using an anti-c-myc antibody (see FIGS. 12 and 13). The data given as percentage values are derived from the comparison of the band intensity of the reference proteins and the band intensity of the synthetic spider silk proteins according to the invention, and are estimated values. Differences in size of the synthetic fibre proteins and reference protein were taken into account. Possible differences in labelling efficiency can be almost precluded.

FIG. 13 shows the heat stability of various synthetic spider silk proteins according to the invention in plant extracts. Surprisingly, the spider silk proteins according to the invention remain in solution even in a prolonged heat treatment of 3 hours (comparison of reference sample R to samples H-60 min, H-120 min and H-180 min). More than 90% of the residual plant proteins are denatured and can be simply separated out via centrifugation (FIG. 13a; comparison of sample R to H-60 min). These unusual properties of the synthetic spider silk proteins according to the invention, which among other things are a consequence of their amino acid sequence and their folding in the plant ER, render possible the development of inexpensive purification strategies that can be realized on a large-scale.

FIG. 14 shows the solubility of synthetic fibre proteins from transgenic plants. In contrast to the bacterially expressed synthetic fibre proteins described in the prior art, the spider silk proteins according to the invention exhibit a surprisingly good solubility in aqueous buffers (R1, R2=Tris buffer, T1, T2=phosphate buffer). These properties also are attributable among other things to the amino acid sequence, and in particular the folding in the endoplasmatic reticulum of plant cells.

### Example 2

### Expression and Stable Accumulation of Synthetic Spider Silk Proteins in the Cell Membrane of Leaves from Transgenic Tobacco and Potato Plants

This example describes the membrane-associated accumulation of spider silk proteins according to the invention in transgenic tobacco and potato plants. In this case, the constructs described in Example 1 that are taken as the basis are used to produce fusion genes, which code for an spider silk protein and for a membrane domain. FIG. 15 shows a general diagram of these constructs. In this case, a NotI fragment was isolated from the plasmid pRT-HOOK, which codes for both the HOOK domain and for a c-myc-immunotag, which then was cloned in spider silk protein gene-carrying derivatives of the pRTA.7/3 vector. Selection of restriction endonuclease recognition sequences at the 5′- and 3′-end of the synthetic spider silk protein genes (SmaI and NaeI) again allows them to be combined with each other in any order, so that larger fibre protein genes can be assimilated in a single cloning step.

FIG. 16 shows the expression of the genes described above in transgenic tobacco and potato plants. As can be seen from a comparison of samples 1, 2 and 3 in this Figure, these transgenic spider silk proteins are not soluble in the aqueous phase in contrast to the proteins according to the invention described in Example 1. This property also can be utilized for the development of purification strategies.

### Example 3

### Targeted Alteration of the Solubility of Spider Silk Proteins by Means of Fusion with Elastin-Like Peptides

In a first step it was shown that fusions with elastin-like peptides also result in an targeted alteration in the solubility behaviour as a function of temperature and concentration even in spider silk proteins expressed in bacteria.

FIG. 5 shows a corresponding expression cassette. Examples for ELP with 10, 20, 30, 40, 60, 70 and 100 pentameric units are identified in the sequences SEQ ID NO: 41 to 47. Examples for DNA sequences and amino acid sequences in the form of the construct SM12-70xELP as the plant expression cassette or as the expression cassette for E. coli are shown in sequences SEQ ID NO: 48-51 or in FIGS. 19 to 22.

FIG. 17 shows the gel electrophoretic analysis of such a purification technique. The spider silk-ELP fusion protein was enriched by heat-treating the crude extract. Surprisingly, the fusion proteins retained the excellent solubility of the spider silk proteins at high temperatures. The bulk of the E. coli proteins were precipitated out at these temperatures.

After concentrating the enriched spider silk protein extract to a high level, the extract was subjected to a temperature of 60° C., after which the ELP spider silk protein precipitated and was removed via pelleting. The pellet was dissolved in water at room temperature, and insoluble components were removed via pelleting.

The spider silk protein fraction was then lyophilised and digested by cyanogen bromide cleavage. The cyanogen bromide cleavage was rendered possible by the methionine residue between the spider silk protein and the ELP peptide.

This was again followed by lyophilisation and dissolution in an aqueous buffer. Concentration to a high level was then performed, wherein the cleaved ELP fragment (ELP(T-R); see FIG. 2) precipitated and was removed via pelleting. The spider silk protein remained in solution (SM12(T-R); see FIG. 17). The solubility was maintained for a prolonged period, for SM12 at 4° C. for 24 h. The identity of spider silk protein purified in this way was demonstrated by the peptide sequencing of the N-terminal end.

In a second step, spider silk proteins were accumulated as ELP fusions in the endo-plasmatic reticulum of transgenic tobacco plants. FIG. 5 also shows the basic structure of these expression cassettes. These fusion proteins having molecular weights of 35,000 Dalton to 100,000 Dalton were all accumulated to high concentrations in plants with an expression level of about 4% of the total soluble protein.

General Molecular Biological Methods

Cloning strategies: Restriction cleavages were performed in 100 μl end volume. As a standard, 10 μg of plasmid DNA, 10 Upper restriction endonuclease, 10 μl of a suitable buffer (10×) were used. DNA fragments were separated from each other via gel electrophoresis, and purified by DNA gel extraction, where necessary. For ligations, the DNA fragment (insert) to be cloned was used in a threefold molar excess to the vector fragment. Sticky-end ligations were performed in one hour, and blunt-end ligations were performed in 12 h at 4° C. with 1 U ligase. The DNA was incorporated both in the cells of E. coli and of A. tumefaciens via electroporation. Transformants were selected on suitable solid nutrient media with the addition of an antibiotic (ampicillin or kanamycin).
PCR: PCR reactions were performed in 50 μl end volume. As a standard, 100 ng of template DNA, 100 pmol of each primer, 1 μl of dNTPs (10 mM) and 5 μl of a suitable buffer were used, along with 1 U Tfl or Taq DNA polymerase. The following conditions were selected for a PCR reaction: 2 min at 95° C., then 30 cycles, each running for 45 sec at 95° C., 45 sec at 50° C. or 55° C., 1 min at 72° C., followed by a cycle for 5 min at 72° C.
Expression and accumulation in tobacco and potato plants: Transgenic plants were selected in an incubator room under uniform illumination at about 20° C. on suitable solid nutrient media containing antibiotic (kanamycin, rifampicin and carbenicillin). After roots appeared, they were allowed to continue growth in pots containing soil in a greenhouse.

As for the rest, the molecular biological and biochemical techniques used in the resent invention can be looked up in available laboratory manuals, e.g., in Sambrook et al. (1989) Molecular Cloning: A Laboratory Manual, 2^{nd} edition, Cold Spring Harbour Laboratory Press, Cold Spring Harbour, New York.

### FIGURES

FIG. 1:

Oligonucleotide sequences that code for spidroin-typical short amino acid repeats.

FIG. 2:

Successive arrangement of oligonucleotide sequences for constructing modules using the DNA sequences of the present invention.

FIG. 3:

Structure of DNA sequences according to the invention made out of modules.

FIG. 4:

Cloning of the gene of the HOOK transmembrane domain with NotI from (pRT-HOOK) in (pRTA.73 syn.spidroin).

FIG. 5:

Diagrammatic representation of the spidroin-ELP expression cassettes. xELP units: 10, 20, 30, 40, 60, 70 or 100 pentamers (Val-Pro-Gly-Val-Gly, SEQ ID NO: 54). The methionine between the spider silk protein and the ELP peptide renders possible the cyanogen bromide cleavage.

FIG. 6:

Change of a base in the BamHI recognition sequence (position 1332) via targeted mutagenesis.

FIG. 7:

Preparation of (pRTRA.73, BamHIA) for directly cloning the synthetic spidroin gene from p9905 or p9609—cancellation of the SmaI recognition sequence (position 463).

FIG. 8:

Introduction of the restriction recognition sequences of SmaI and NaeI into the vector (pRTRA.73, BamHIA+SmaIA) for cloning synthetic spidroin genes.

FIG. 9:

General depiction of the manufacture of transgenic plants producing spider silk protein.

FIG. 10:

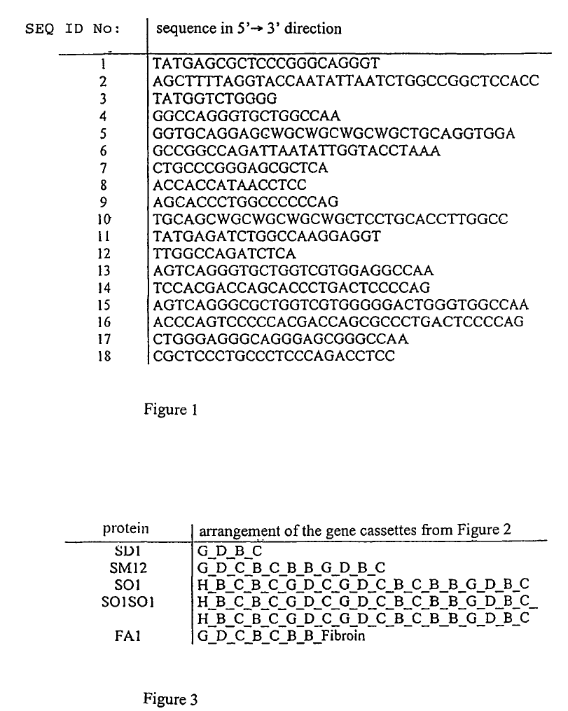

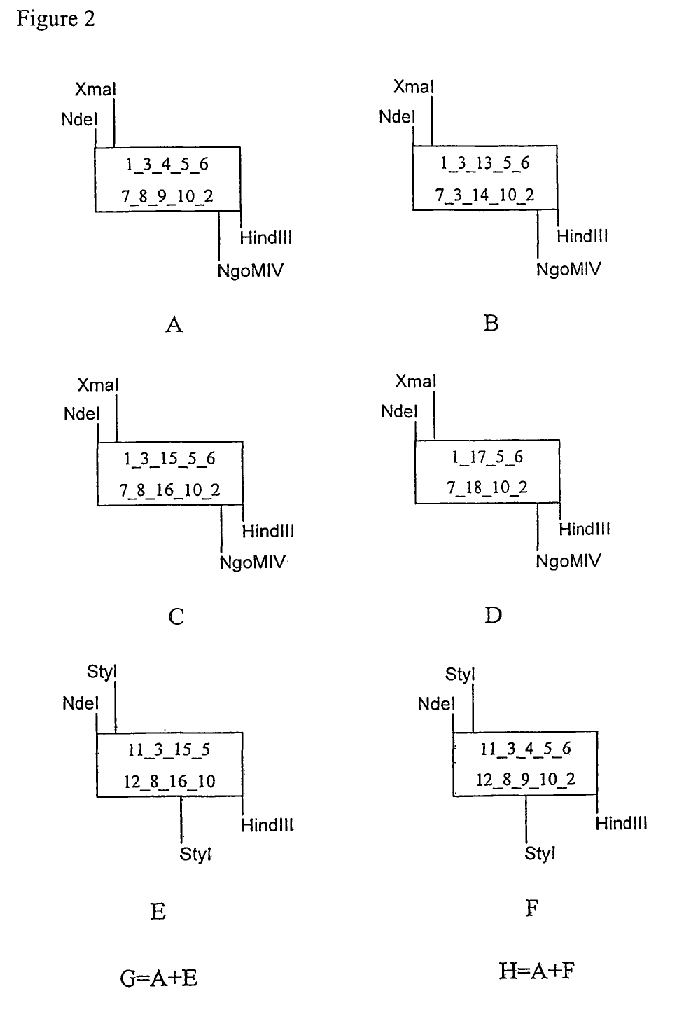

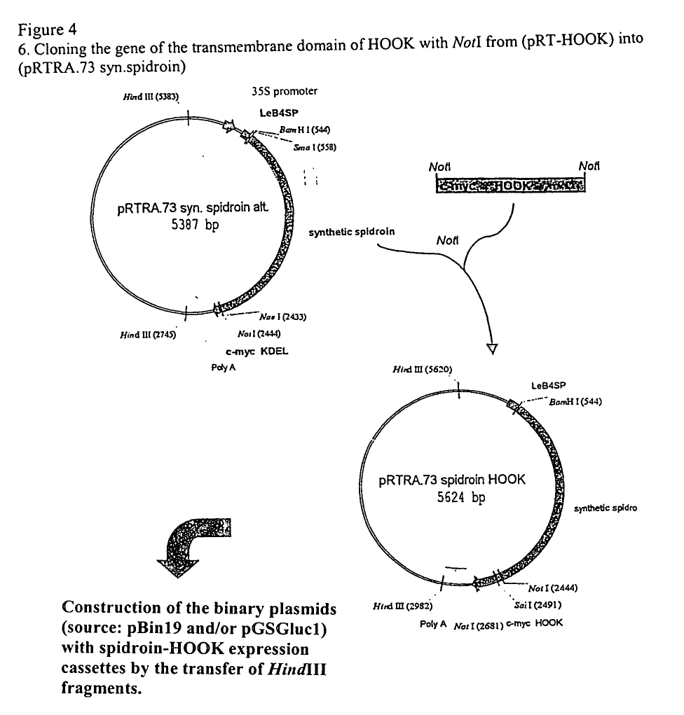

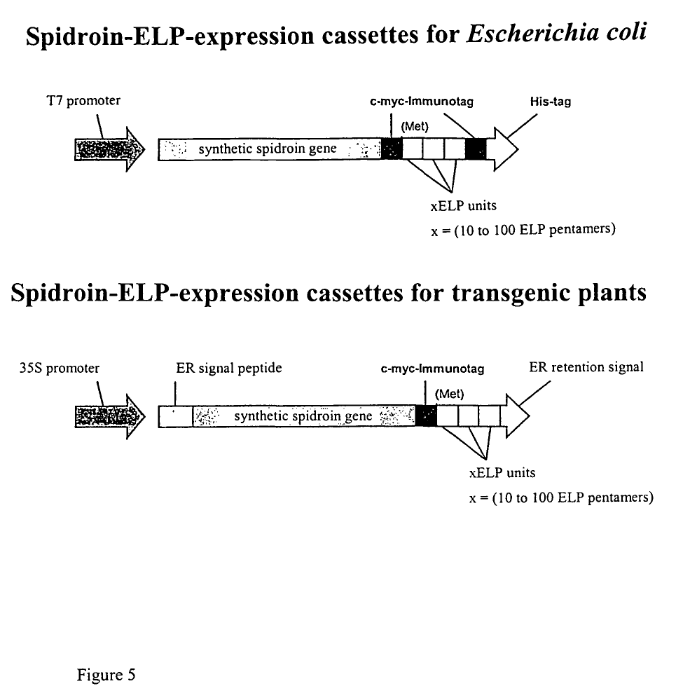

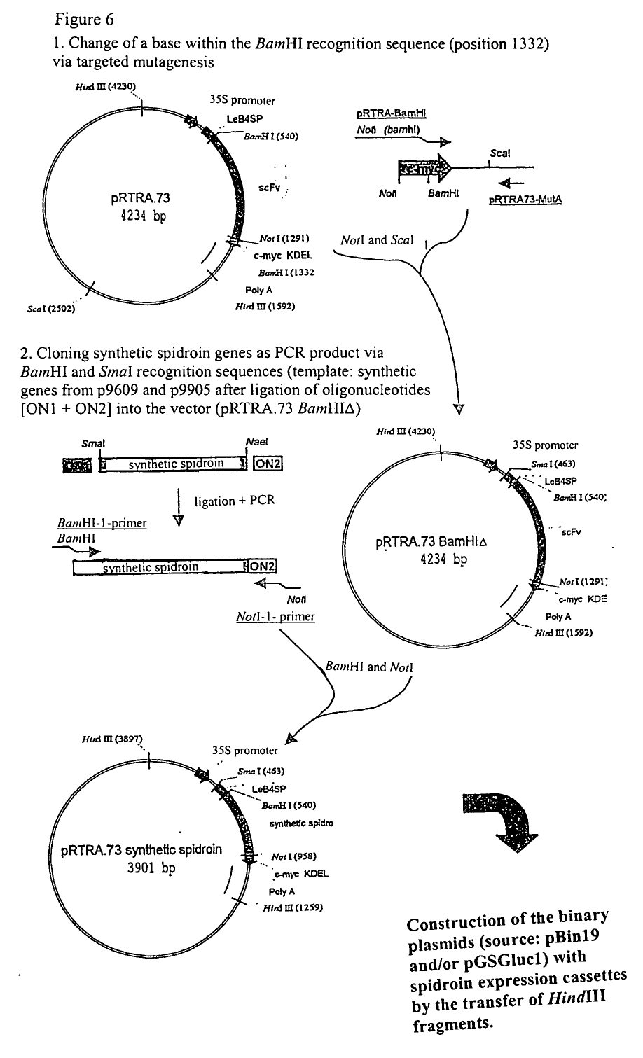

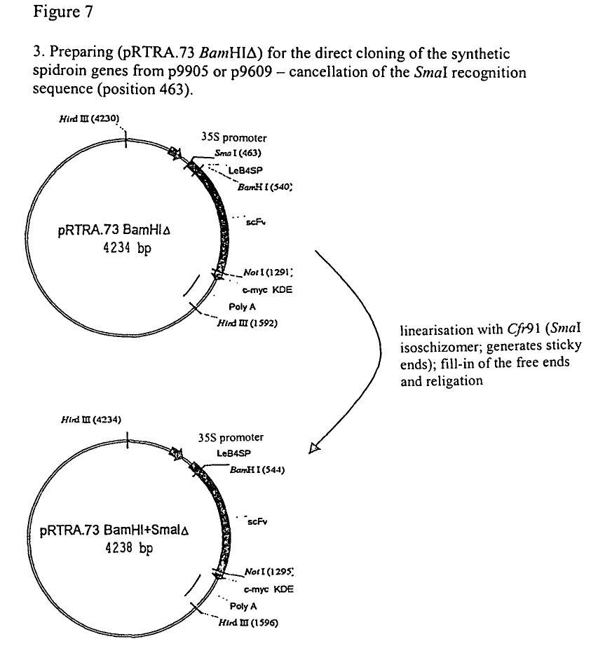

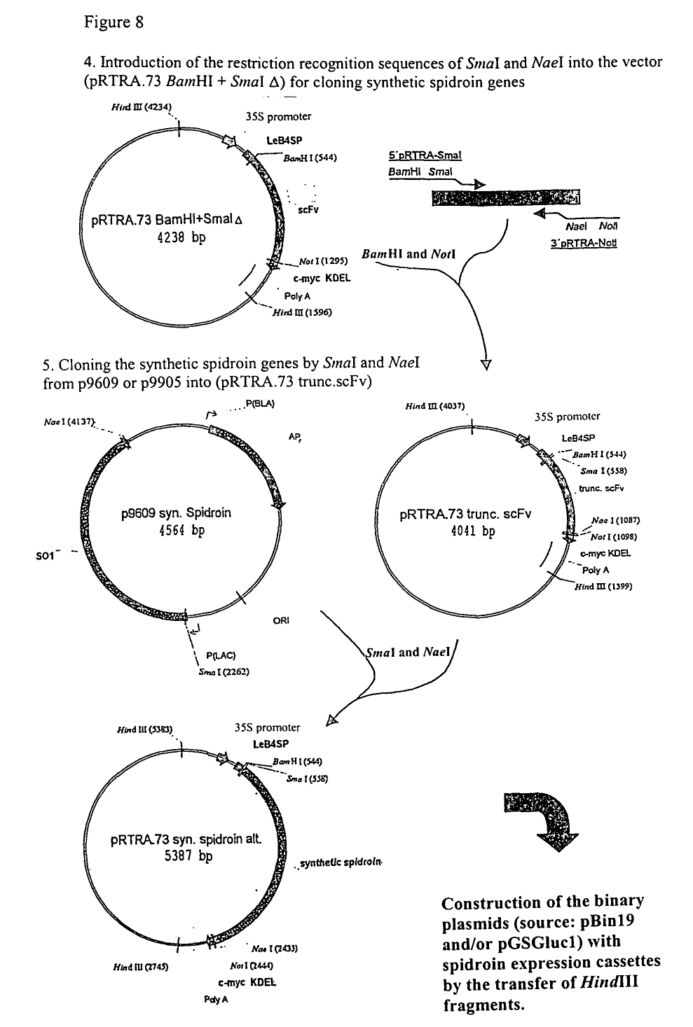

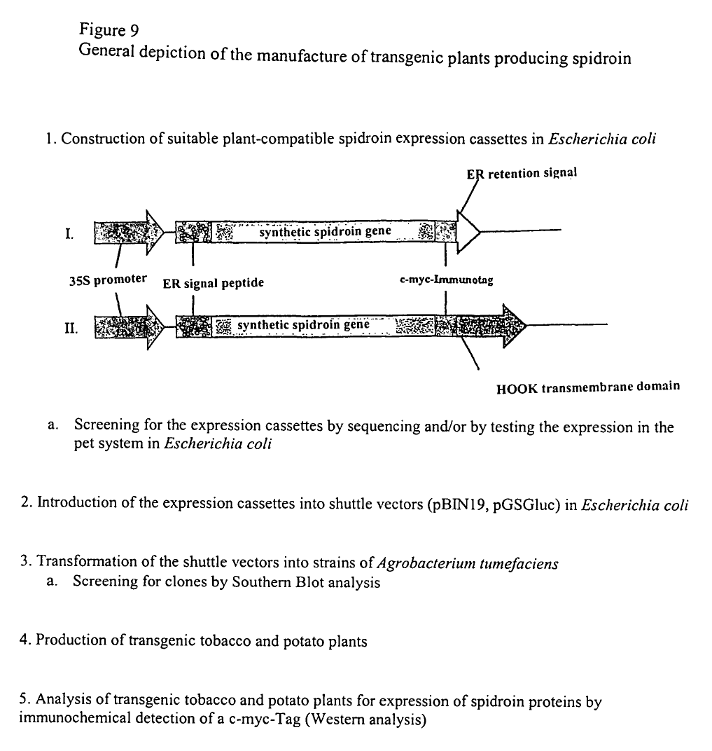

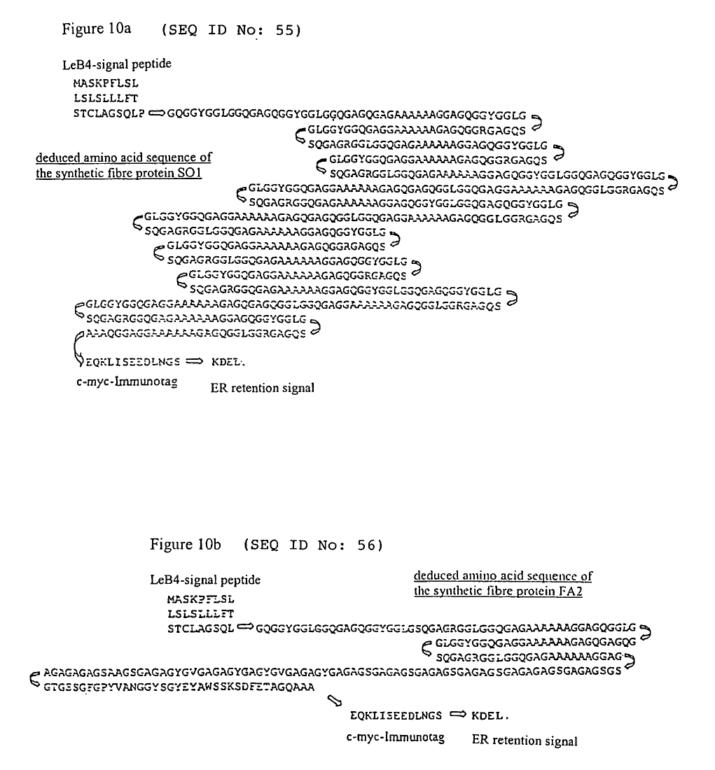

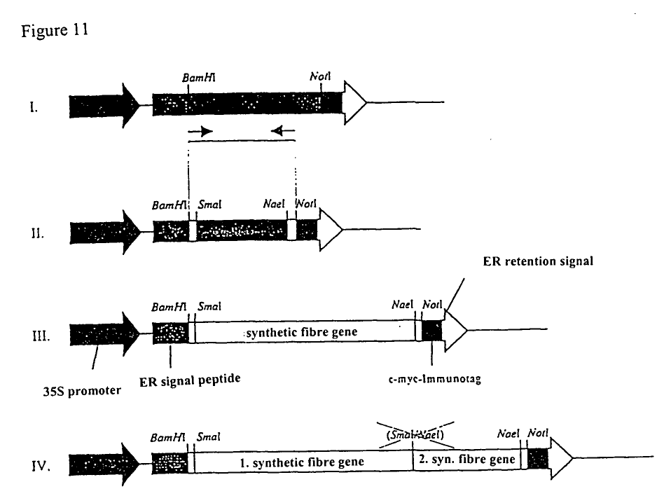

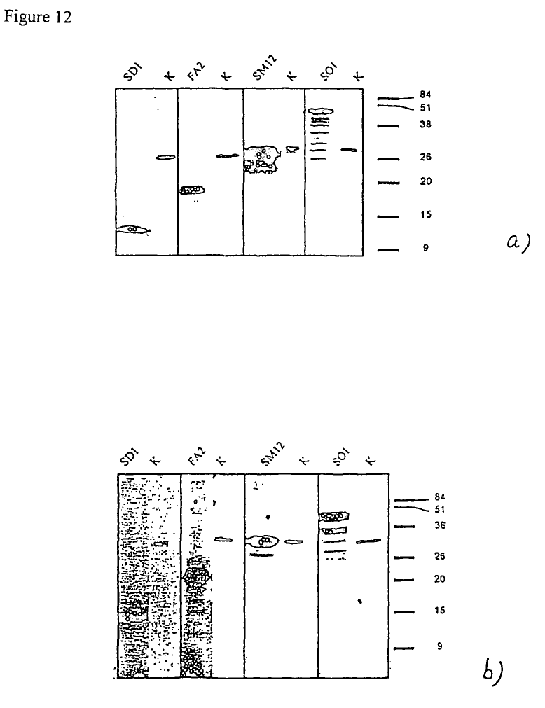

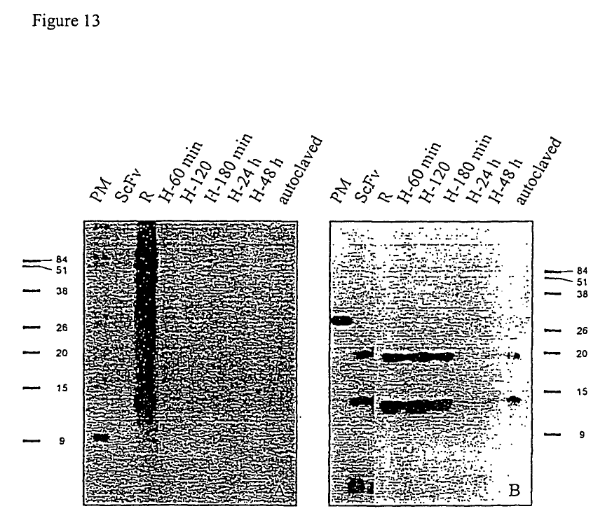

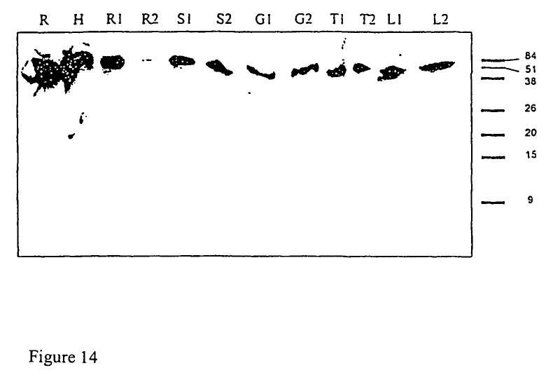

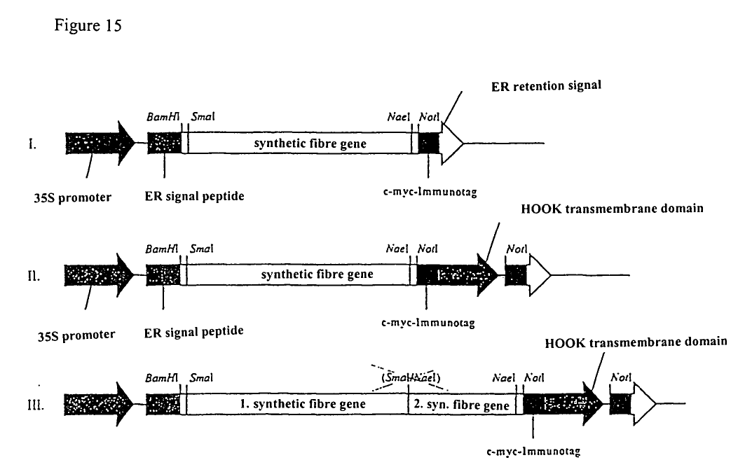

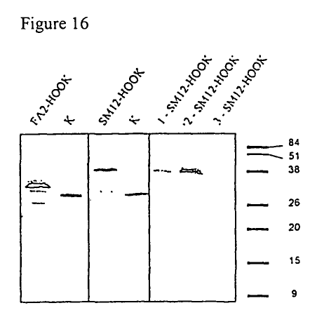

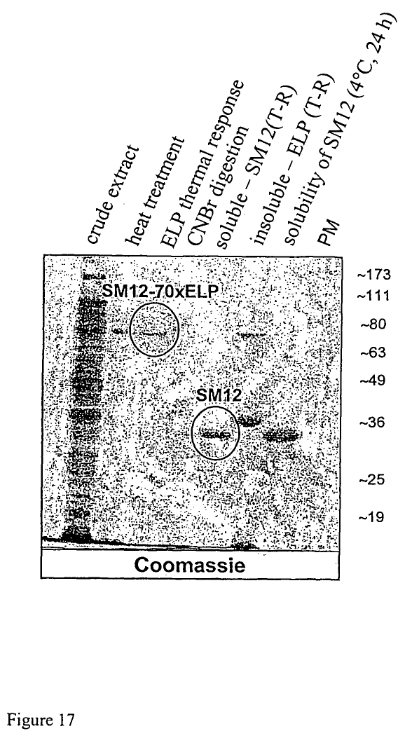

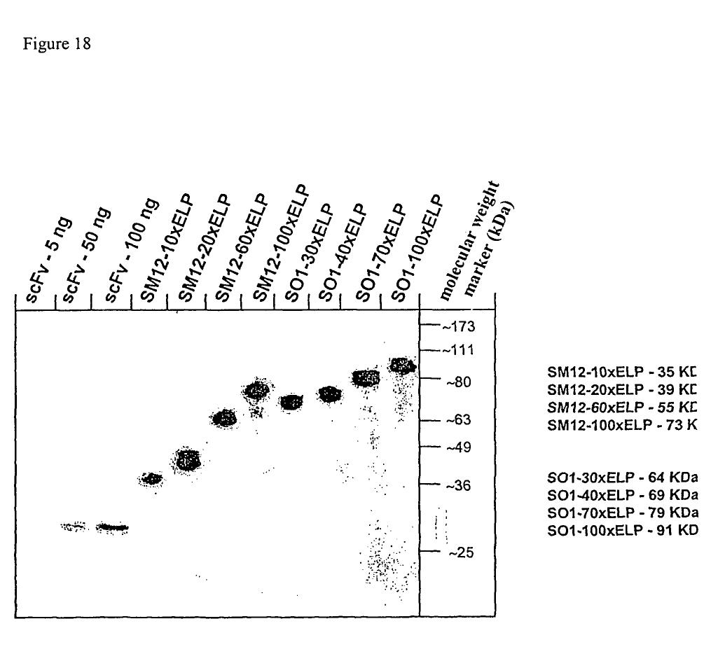

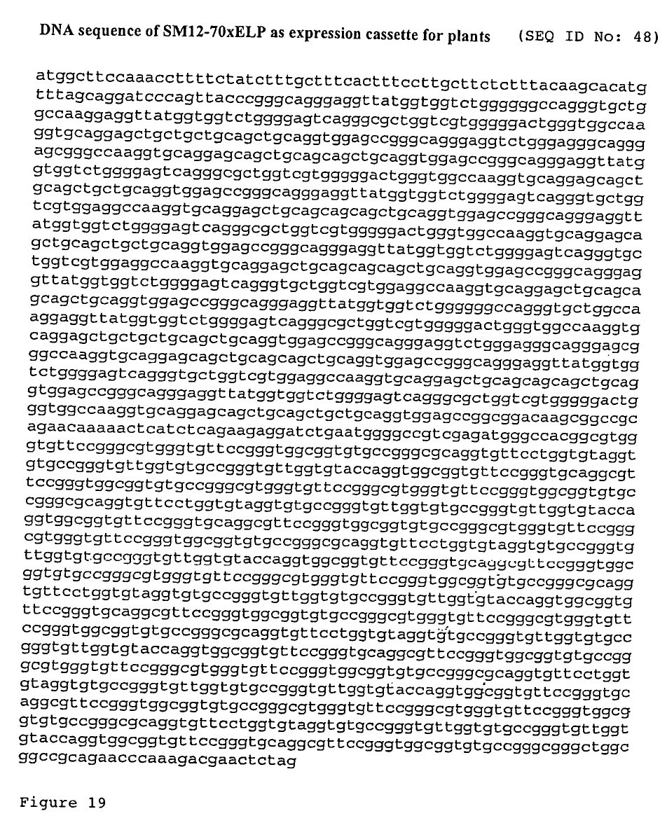

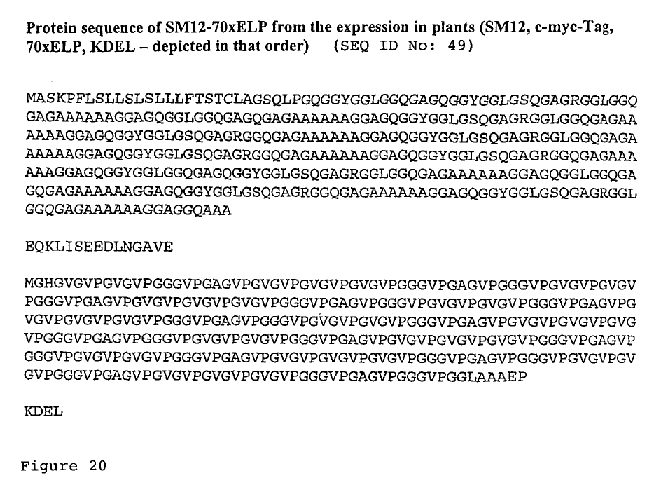

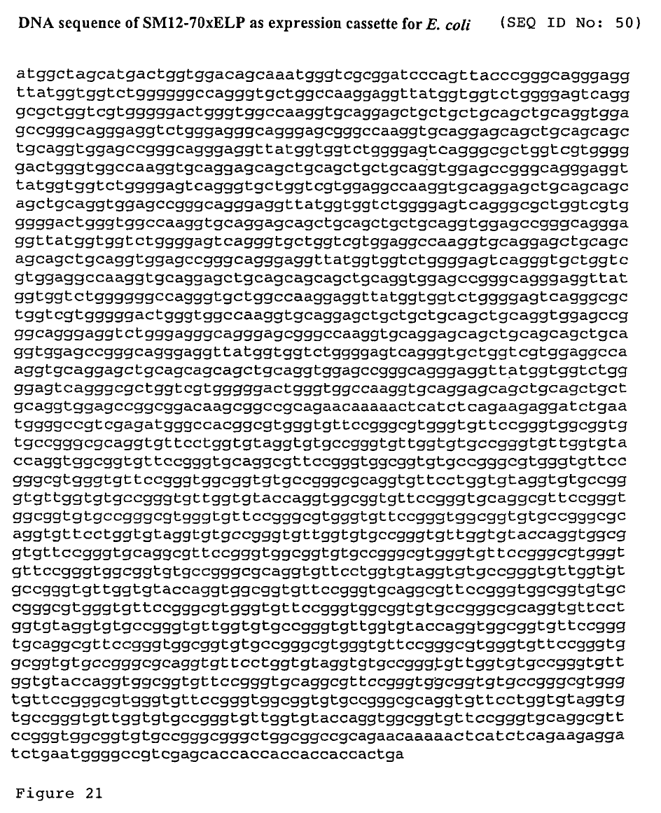

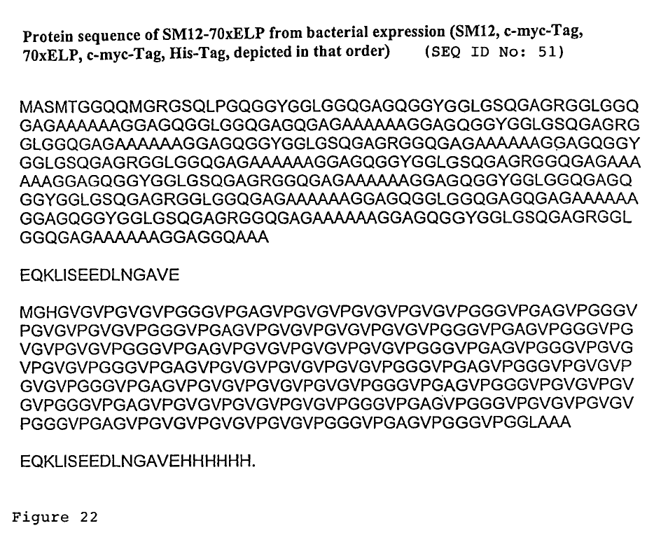

(a) Depiction of the modular structure of the spider silk proteins according to the invention based on the example of the SO1 sequence. Amino acids 1-28: LeB4 signal peptide; amino acids 29-659: synthetic spider silk protein sequence; amino acids 660-672: c-myc-tag; amino acids 673-676: ER retention signal.

Arrangement of the sequence modules according to the original sequence specified in Simmons et al., “Molecular orientation and two-component nature of the crystalline fraction of spider dragline silk” (1996), Science 271: 84-87.

(b) Depiction of the modular structure of the synthetic fibre hybrid protein FA2. Amino acids 1-27: LeB4 signal peptide; amino acids 28-130: synthetic fibre protein sequence of the spider; amino acids 131-247: synthetic fibre protein sequence of the silkworm; amino acids 248-260: c-myc-tag; amino acids 261-264: ER retention signal.

FIG. 11:

Diagrammatic representation of the construction of gene cassettes for the accumulation of synthetic fibre proteins of the spider and silkworm in the ER of transgenic plants.

FIG. 12:

(a) Expression of synthetic fibre proteins of the spider (SD 1, SM12, SO1) or the hybrid of spider and silkworm (FA2) in leaves of transgenic tobacco plants. 40 μg of total protein were analysed in SDS sample buffer. SD1: 13 kDa; FA2: 20 kDa; SM12: 31 kDa; SO1: 52 kDa; K: positive control 50 ng ScFv.

(b) Expression of the synthetic fibre proteins of the spider (SD 1, SM12, SO1) or hybrid of spider and silkworm (FA2) in transgenic potato plants.

40 μg of total protein were also analysed in the SDS sample buffer. SD1:1:3 kDa; FA2: 20 kDa; SM12: 31 kDa; SO1: 52 kDa; K: positive control 50 ng ScFv.

FIG. 13:

Depiction of the heat resistance of the synthetic fibre proteins of the spider and silkworm based on the constructs SD1 and FA2. A: Coomassie-stained gel. B: Immunochemical detection of the synthetic fibre proteins SD1 and FA2 via anti-c-myc antibodies. PM: protein marker; ScFv: 50 ng ScFv; R: aqueous plant extract from leaves of transgenic plants for SDI and FA2; H: heating step 60 min, 120 min, 180 min, 24 h and 48 h at 90° C.

Plant extract constituents precipitated during heat treatment were separated by centrifugation.

FIG. 14:

Analysis of the solution properties and stability of the synthetic spider silk protein SO1 after ammonium sulfate precipitation.

10 g of leaf material were shock-frozen in liquid nitrogen, triturated, taken up in 20 ml of crude extract buffer, shaken for 30 min at 38° C., and then insoluble components have been removed via centrifugation (30 min, 10,000 rpm). The supernatant (R) was then heated to 90° C. for 10 min, and the precipitate was removed via centrifugation (30 min, 10,000 rpm). Ammonium sulfate saturated up to a concentration of 20% in the final volume was added to the supernatant (H), the mixture was stirred by rotation at room temperature for 4 h, and the precipitate was then removed via centrifugation for 60 min at 4000 rpm and 4° C. After that ammonium sulfate was added to the supernatant up to a concentration of 30% saturation and the mixture was agitated overnight at room temperature. The solution was split into 5 aliquots, and the precipitate was removed by centrifugation (60 min, 4000 rpm, 4° C.). The supernatants were discarded, and the remaining pellets were taken up in the following solutions: R1: crude extract buffer (50 mM Tris/HCl pH 8.0; 100 mM NaCl, 10 mM MgSO₄); S: SDS sample buffer; G: 0.1 M phosphate buffer, 0.01 M Tris/HCl, 6 M guanidinium hydrochloride/HCl pH 6.5; T: 1×PBS, 1% TritonX-100; L: LiBr.

The charges were shaken for 1 h at 37° C., and insoluble components were removed by centrifugation (30 min, 10,000 rpm). An aliquot of each charge was then removed in order to prepare SDS gel electrophoresis (R1, S1, G1, T1, L1). The charges were allowed to stand at room temperature for 36 h. Insoluble components were removed via centrifugation (30 min, 10,000 rpm). An aliquot of each charge was again removed and prepared for SDS gel electrophoresis (R2, S2, G2, T2, L2). Comparable volumes were again analyzed.

FIG. 15:

Diagrammatic view of the construction of gene cassettes for the accumulation of cell membraneous synthetic fibre proteins of the spider and silkworm in transgenic plants.

FIG. 16:

Expression of the fibre fusion proteins SM12-HOOK, SO1-HOOK and FA2-HOOK in the leaves of transgenic potato plants.

FIG. 17:

Gel electrophoretic analysis of the enrichment of bacterially expressed spider silk proteins after fusion with ELPs. Spider silk protein: 30,000 Dalton.

FIG. 18:

Western blot analysis of the expression of spider silk-ELP fusion proteins in transgenic tobacco plants. 2.5 μg of the total plant protein were separated, and the spider silk proteins were detected on the Western blot by ECL. The spider silk protein concentration was estimated to be at least 4% of the total soluble protein by comparing it with the standard.

FIG. 19:

DNA sequence of SM12-70xELP as the plant expression cassette.

FIG. 20:

Protein sequence of SM12-70xELP from plant expression (SM12, c-myc-tag, 70xELP, KDEL (SEQ ID NO: 52)—depicted in that order).

FIG. 21:

DNA sequence of SM12-70xELP as expression cassette for E. coli.

FIG. 22:

Protein sequence of SM12-70xELP from bacterial expression (SM12, c-myc-tag, 70xELP, c-myc-tag, HisTag—depicted in that order).

1-37. (canceled)

38. A DNA sequence that codes for a synthetic spider silk protein having a homology of at least 80% to the spidroin 1 protein of Nephila clavipes, wherein the DNA sequence comprises modules comprising a group of successively arranged oligonucleotide sequences, wherein the oligonucleotide sequences each code for repetitive units from spidroin proteins, wherein the modules are freely arranged, and wherein the oligonucleotide sequences are selected from the group consisting of:
a) TATGAGCGCTCCCGGGCAGGGT (SEQ ID NO: 1);
b) AGCTTTTAGGTACCAATATTAATCTGGCCGGCTCCACC (SEQ ID NO: 2);
c) TATGGTCTGGGG (SEQ ID NO: 3);
d) GGCCAGGGTGCTGGCCAA (SEQ ID NO: 4);
e) GGTGCAGGAGCWGCWGCWGCWGCTGCAGGTGGA (SEQ ID NO: 5);
f) GCCGGCCAGATTAATATTGGTACCTAAA (SEQ ID NO: 6);
g) CTGCCCGGGAGCGCTCA (SEQ ID NO: 7);
h) ACCACCATAACCTCC (SEQ ID NO: 8);
i) AGCACCCTGGCCCCCCAG (SEQ ID NO: 9);
j) TGCAGCWGCWGCWGCWGCTCCTGCACCTTGGCC (SEQ ID NO: 10);
k) TATGAGATCTGGCCAAGGAGGT (SEQ ID NO: 11);
l) TTGGCCAGATCTCA (SEQ ID NO: 12);
m) AGTCAGGGTGCTGGTCGTGGAGGCCAA (SEQ ID NO: 13);
n) TCCACGACCAGCACCCTGACTCCCCAG (SEQ ID NO: 14);
o) AGTCAGGGCGCTGGTCGTGGGGGACTGGGTGGCCAA (SEQ ID NO: 15);
p) ACCCAGTCCCCCACGACCAGCGCCCTGACTCCCCAG (SEQ ID NO: 16);
q) CTGGGAGGGCAGGGAGCGGGCCAA (SEQ ID NO: 17);
r) CGCTCCCTGCCCTCCCAGACCTCC (SEQ ID NO: 18); and
s) sequences that exhibit at least 80% sequence identity to the sequences a) to r).

39. The DNA sequence according to claim 38, wherein the modules comprise at least 4 oligonucleotide sequences.

40. The DNA sequence according to claim 38, wherein the DNA sequence comprises at least 4 modules.

41. The DNA sequence according to claim 38, wherein the DNA sequence further comprises nucleic acid sequences that code for repetitive units from fibroin proteins.

42. The DNA sequence according to claim 38 comprising one of the sequences identified in SEQ ID NO. 19 to 29.

43. A recombinant nucleic acid molecule comprising
a) a DNA sequence that codes for a synthetic spider silk protein wherein the DNA sequence comprises modules comprising a group of successively arranged oligonucleotide sequences, wherein the oligonucleotide sequences each code for repetitive units from spidroin proteins, wherein the modules are freely arranged such that it is possible for the synthetic spider silk protein to exhibit an altered range of properties in comparison to native spider silk protein,
b) an ubiquitously acting promoter, and
c) a nucleic acid sequence coding for ELPs.

44. The recombinant nucleic acid molecule according to claim 43, wherein the DNA sequence is a DNA sequence according to claim 38.

45. The recombinant nucleic acid molecule according to claim 43 further comprising at least one nucleic acid sequence that codes for a plant signal peptide.

46. The recombinant nucleic acid molecule according to claim 45, wherein the plant signal peptide mediates the transport into the endoplasmatic reticulum (ER).

47. The recombinant nucleic acid molecule according to claim 45, wherein the nucleic acid sequence that codes for the plant signal peptide is an LeB4Sp sequence.

48. The recombinant nucleic acid molecule according to claim 43 further comprising a nucleic acid sequence that codes for an ER retention peptide.

49. The recombinant nucleic acid molecule according to claim 11, wherein the ER retention peptide comprises the KDEL sequence (SEQ ID NO: 52).

50. The recombinant nucleic acid molecule according to claim 43 further comprising a nucleic acid sequence that codes for a transmembrane domain.

51. The recombinant nucleic acid molecule according to claim 50, wherein the nucleic acid sequence codes for the transmembrane domain of the PDGF receptor.

52. The recombinant nucleic acid molecule according to claim 43, wherein the ELPs comprise from 10 to 100 pentameric units.

53. The recombinant nucleic acid molecule according to claim 43 comprising one of the sequences identified in SEQ ID NO. 48 and 50.

54. A microorganism comprising a recombinant nucleic acid molecule according to claim 43.

55. A recombinant spider silk protein coded by a DNA sequence according to claim 38.

56. The spider silk protein according to claim 55, wherein the protein's molecular weight ranges from 10 to 160 kDa.

57. A recombinant spider silk protein comprising one of the amino acid sequences identified in SEQ ID NO. 30 to 40.

58. A method of manufacturing spider silk protein-producing plants or plant cells comprising the following steps:
a) manufacturing a recombinant nucleic acid molecule according to claim 43;
b) transferring the nucleic acid molecule from a) to plant cells; and
c) optionally, regenerating plants from the transformed plant cells.

59. Transgenic plant cells comprising a recombinant nucleic acid molecule according to claim 43.

60. Transgenic plants comprising a plant cell according to claim 59 as well as parts of these plants, transgenic harvest products and transgenic propagating material of these plants, such as protoplasts, plant cells, calli, seeds, tubers, cuttings, and the transgenic progeny of these plants.

61. The transgenic plants according to claim 60 selected from the group consisting of tobacco plants and potato plants.

62. A method of obtaining plant spider silk protein comprising the following steps:
a) transferring a recombinant nucleic acid molecule according to claim 43 to plant cells;
b) optionally, regenerating plants from the transformed plant cells; and
c) processing the plant cells from a) or plants from b) to obtain plant spider silk protein.

63. A method of obtaining recombinant manufactured spider silk protein comprising the following steps:
a) transferring a recombinant nucleic acid molecule according to claim 43 to cells; and
b) purifying the spider silk protein by heat-treating the cell extract and then separating the denatured proteins naturally occurring in the cell.

64. A method of obtaining recombinant manufactured spider silk protein comprising the following steps:
a) transferring a recombinant nucleic acid molecule according to claim 43 to cells; and
b) purifying the spider silk protein by adjusting an acidic pH by adding acid to the cell extract and then separating the denatured proteins naturally occurring in the cell.

65. The method according to claim 64, wherein the acidic pH ranges from 2.5 to 3.5.

66. The method according to claim 64, wherein the acid is hydrochloric acid.

67. A method of obtaining recombinant manufactured spider silk protein comprising the following steps:
a) transferring a recombinant nucleic acid molecule according to claim 43 to cells; and
b) purifying the spider silk protein as follows:
i) enriching the spider silk-ELP fusion protein by heat-treating the cell extract;
ii) precipitating the spider silk-ELP fusion protein by further increasing the temperature; and
iii) cleaving off the ELP fragment.

68. The method of claim 67, wherein the temperature is at least 60° C.

69. The method of claim 67, wherein the ELP fragment is cleaved off via digestion with CNBr.

70. The method according to claim 63, wherein the cells are selected from the group consisting of plant cells, animal cells and bacterial cells.

71. The method according to claim 64, wherein the cells are selected from the group consisting of plant cells, animal cells and bacterial cells.

72. The method according to claim 67, wherein the cells are selected from the group consisting of plant cells, animal cells and bacterial cells.

73. A method of manufacturing synthetic threads, films and/or membranes from spider silk proteins derived from a DNA sequence according to claim 38.

74. The method according to claim 73, wherein the threads, films and/or membranes are used for medical purposes.

75. The method according to claim 74, wherein the threads, films and/or membranes are used for closing wounds and/or as frames or covers for artificial organs.

76. The method according to claim 73, wherein the films and/or membranes are used as adhesion surfaces for cultivated cells and/or for filtering purposes.

77. The DNA sequence according to claim 38, wherein at least one property of the synthetic spider silk protein selected from the group consisting of tensile strength, elasticity, swelling capacity, solubility behaviour, acid stability and heat resistance is altered compared to a native spider silk protein.


SEQUENCE LISTING (51 sequences)

SEQ ID NO: 1 - DNA, 22 bp, artificial sequence
  description of the artificial sequence repetitive unit from spidroin proteins
tatgagcgctcccgggcagggt

SEQ ID NO: 2 - DNA, 38 bp, artificial sequence
  description of the artificial sequence repetitive unit from spidroin proteins
agcttttaggtaccaatattaatctggccggctccacc

SEQ ID NO: 3 - DNA, 12 bp, artificial sequence
  description of the artificial sequence repetitive unit from spidroin proteins
tatggtctgggg

SEQ ID NO: 4 - DNA, 18 bp, artificial sequence
  description of the artificial sequence repetitive unit from spidroin proteins
ggccagggtgctggccaa

SEQ ID NO: 5 - DNA, 33 bp, artificial sequence
  description of the artificial sequence repetitive unit from spidroin proteins
ggtgcaggagcwgcwgcwgcwgctgcaggtgga

SEQ ID NO: 6 - DNA, 28 bp, artificial sequence
  description of the artificial sequence repetitive unit from spidroin proteins
gccggccagattaatattggtacctaaa

SEQ ID NO: 7 - DNA, 17 bp, artificial sequence
  description of the artificial sequence repetitive unit from spidroin proteins
ctgcccgggagcgctca

SEQ ID NO: 8 - DNA, 15 bp, artificial sequence
  description of the artificial sequence repetitive unit from spidroin proteins
accaccataacctcc

SEQ ID NO: 9 - DNA, 18 bp, artificial sequence
  description of the artificial sequence repetitive unit from spidroin proteins
agcaccctggccccccag

SEQ ID NO: 10 - DNA, 33 bp, artificial sequence
  description of the artificial sequence repetitive unit from spidroin proteins
tgcagcwgcwgcwgcwgctcctgcaccttggcc

SEQ ID NO: 11 - DNA, 22 bp, artificial sequence
  description of the artificial sequence repetitive unit from spidroin proteins
tatgagatctggccaaggaggt

SEQ ID NO: 12 - DNA, 14 bp, artificial sequence
  description of the artificial sequence repetitive unit from spidroin proteins
ttggccagatctca

SEQ ID NO: 13 - DNA, 27 bp, artificial sequence
  description of the artificial sequence repetitive unit from spidroin proteins
agtcagggtgctggtcgtggaggccaa

SEQ ID NO: 14 - DNA, 27 bp, artificial sequence
  description of the artificial sequence repetitive unit from spidroin proteins
tccacgaccagcaccctgactccccag

SEQ ID NO: 15 - DNA, 36 bp, artificial sequence
  description of the artificial sequence repetitive unit from spidroin proteins
agtcagggcgctggtcgtgggggactgggtggccaa

SEQ ID NO: 16 - DNA, 36 bp, artificial sequence
  description of the artificial sequence repetitive unit from spidroin proteins
acccagtcccccacgaccagcgccctgactccccag

SEQ ID NO: 17 - DNA, 24 bp, artificial sequence
  description of the artificial sequence repetitive unit from spidroin proteins
ctgggagggcagggagcgggccaa

SEQ ID NO: 18 - DNA, 24 bp, artificial sequence
  description of the artificial sequence repetitive unit from spidroin proteins
cgctccctgccctcccagacctcc

SEQ ID NO: 19 - DNA, 327 bp, artificial sequence
  description of the artificial sequence construct SB1
ggatcccagttagggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccggacaagcggccgca

SEQ ID NO: 20 - DNA, 705 bp, artificial sequence
  description of the artificial sequence construct SE1
ggatcccagttagggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccggacaagcggccgca

SEQ ID NO: 21 - DNA, 426 bp, artificial sequence
  description of the artificial sequence construct SD1
ggatcccagttagggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggacaagcggccgca

SEQ ID NO: 22 - DNA, 3783 bp, artificial sequence
  description of the artificial sequence construct SO1SO1
ggatcccagttacccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgca

SEQ ID NO: 23 - DNA, 2985 bp, artificial sequence
  description of the artificial sequence construct SO1SM12
ggatcccagttacccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgca

SEQ ID NO: 24 - DNA, 5658 bp, artificial sequence
  description of the artificial sequence construct SO1SO1SO1
ggatcccagttacccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgca

SEQ ID NO: 25 - DNA, 672 bp, artificial sequence
  description of the artificial sequence construct FA2
ggatcccagttagggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggtccggaagtggtgcaggtgccggaagcggagcaggagccggtgccggatctggtgccggtgccggaagcggtgctggtgccggaagcggtgctggtgccggatcaggagcgggtgccggttatggtgcgggagccggtgttgggtacggagccggttatggagcgggagccggtgttgggtacggagccggtgcaggttccggggccgcaagcggcgcaggagccggtgccggagctgggacagggagttcaggatttgggccctacgttgcaaatggtggttattcaggctatgaatacgcgtggagtagtaagtctgattttgagactgccggacaagcggccgca

SEQ ID NO: 26 - DNA, 525 bp, artificial sequence
  description of the artificial sequence construct SA1
ggatcccagttagggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggacaagcggccgca

SEQ ID NO: 27 - DNA, 1908 bp, artificial sequence
  description of the artificial sequence construct SO1
ggatcccagttacccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgca

SEQ ID NO: 28 - DNA, 1110 bp, artificial sequence
  description of the artificial sequence construct SM12
ggatcccagttacccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgca

SEQ ID NO: 29 - DNA, 831 bp, artificial sequence
  description of the artificial sequence construct SF1
ggatcccagttacccgggcagggaggttatggtggtctggggggccagggtgctggccaaggaggttatggtggtctggggggccagggtgctggccaaggtgcaggagctgctgctgcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccggcggacaagcggccgca

SEQ ID NO: 30 - protein, 104 aa, artificial sequence
  description of the artificial sequence SB1 protein
GQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQAA

SEQ ID NO: 31 - protein, 230 aa, artificial sequence
  description of the artificial sequence SE1 protein
GQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQAA

SEQ ID NO: 32 - protein, 137 aa, artificial sequence
  description of the artificial sequence SD1 protein
GQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGQAA

SEQ ID NO: 33 - protein, 1255 aa, artificial sequence
  description of the artificial sequence SO1SO1 protein
GQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAA

SEQ ID NO: 34 - protein, 989 aa, artificial sequence
  description of the artificial sequence SO1SM12 protein
GQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAA

SEQ ID NO: 35 - protein, 1880 aa, artificial sequence
  description of the artificial sequence SO1SO1SO1 protein
GQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAA

SEQ ID NO: 36 - protein, 219 aa, artificial sequence
  description of the artificial sequence FA2 protein
GQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGSGSGAGAGSGAGAGAGSGAGAGSGAGAGSGAGAGSGAGAGYGAGAGVGYGAGYGAGAGVGYGAGAGSGAASGAGAGAGAGTGSSGFGPYVANGGYSGYEYAWSSKSDFETAGQAA

SEQ ID NO: 37 - protein, 170 aa, artificial sequence
  description of the artificial sequence SA1 protein
GQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQAA

SEQ ID NO: 38 - protein, 630 aa, artificial sequence
  description of the artificial sequence SO1 protein
GQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAA

SEQ ID NO: 39 - protein, 364 aa, artificial sequence
  description of the artificial sequence SM12 protein
GQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAA

SEQ ID NO: 40 - protein, 271 aa, artificial sequence
  description of the artificial sequence SF1 protein
GQGGYGGLGGQGAGQGGYGGLGGQGAGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGGQAA

SEQ ID NO: 41 - DNA, 182 bp, artificial sequence
  description of the artificial sequence ELP containing 10 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 42 - DNA, 332 bp, artificial sequence
  description of the artificial sequence ELP containing 20 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 43 - DNA, 482 bp, artificial sequence
  description of the artificial sequence ELP containing 30 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 44 - DNA, 632 bp, artificial sequence
  description of the artificial sequence ELP containing 40 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 45 - DNA, 932 bp, artificial sequence
  description of the artificial sequence ELP containing 60 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 46 - DNA, 1082 bp, artificial sequence
  description of the artificial sequence ELP containing 70 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 47 - DNA, 1532 bp, artificial sequence
  description of the artificial sequence ELP containing 100 pentameric units
ctcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgc

SEQ ID NO: 48 - DNA, 2322 bp, artificial sequence
  description of the artificial sequence SM12-70xELP (plants)
atggcttccaaaccttttctatctttgctttcactttccttgcttctctttacaagcacatgtttagcaggatcccagttacccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgcagaacaaaaactcatctcagaagaggatctgaatggggccgtcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgcagaacccaaagacgaactctag

SEQ ID NO: 49 - protein, 773 aa, artificial sequence
  description of the artificial sequence SM12-70xELP (plants)
MASKPFLSLLSLSLLLFTSTCLAGSQLPGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAAAEQKLISEEDLNGAVEMGHGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGGLAAAEPKDEL

SEQ ID NO: 50 - DNA, 2334 bp, artificial sequence
  description of the artificial sequence SM12-70xELP (E.coli)
atggctagcatgactggtggacagcaaatgggtcgcggatcccagttacccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccgggcagggaggttatggtggtctggggagtcagggtgctggtcgtggaggccaaggtgcaggagctgcagcagcagctgcaggtggagccgggcagggaggttatggtggtctggggagtcagggtgctggtcgtggaggccaaggtgcaggagctgcagcagcagctgcaggtggagccgggcagggaggttatggtggtctggggggccagggtgctggccaaggaggttatggtggtctggggagtcagggcgctggtcgtgggggactgggtggccaaggtgcaggagctgctgctgcagctgcaggtggagccgggcagggaggtctgggagggcagggagcgggccaaggtgcaggagcagctgcagcagctgcaggtggagccgggcagggaggttatggtggtctggggagtcagggtgctggtcgtggaggccaaggtgcaggagctgcagcagcagctgcaggtggagccgggcagggaggttatggtggtctggggagtcagggcgctggtcgtgggggactgggtggccaaggtgcaggagcagctgcagctgctgcaggtggagccggcggacaagcggccgcagaacaaaaactcatctcagaagaggatctgaatggggccgtcgagatgggccacggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgggctggcggccgcagaacaaaaactcatctcagaagaggatctgaatggggccgtcgagcaccaccaccaccaccactga

SEQ ID NO: 51 - protein, 777 aa, artificial sequence
  description of the artificial sequence SM12-70xELP (E.coli)
MASMTGGQQMGRGSQLPGQGGYGGLGGQGAGQGGYGGLGSQGAGRGGLGGQGAGAAAAAAGGAGQGGLGGQGAGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGLGGQGAGAAAAAAGGAGQGGYGGLGSQGAGRGGQGAGAAAAAAGGAGQGGYGGLGSQGAGRGGQGAGAAAAAAGGAGQGGYGGLGGQGAGQGGYGGLGSQGAGRGGLGGQGAGAAAAAAGGAGQGGLGGQGAGQGAGAAAAAAGGAGQGGYGGLGSQGAGRGGQGAGAAAAAAGGAGQGGYGGLGSQGAGRGGLGGQGAGAAAAAAGGAGGQAAAEQKLISEEDLNGAVEMGHGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGGLAAAEQKLISEEDLNGAVEHHHHHH